(19) United States
(12) Patent Application Publication
(10) Pub. No.: US 2017/0266174 A1
(43) Pub. Date: Sep. 21, 2017

(54) Compositions and Methods for Extending Lifespan

(71) Applicant: COLLABORATIVE MEDICINAL DEVELOPMENT LLC, Sausalito, CA (US)

(72) Inventors: Ashley Bush, Melbourne, Victoria (AU); Gawain McColl, Nunawading, Victoria (AU)

(73) Assignee: COLLABORATIVE MEDICINAL DEVELOPMENT LLC, Sausalito, CA (US)

(21) Appl. No.: 15/505,384

(22) PCT Filed: Aug. 21, 2015
(86) PCT No.: PCT/US15/46377

§ 371 Date: Feb. 21, 2017

Related U.S. Application Data
(60) Provisional application No. 62/040,395, filed on Aug. 21, 2014.

(51) Int. Cl. A61K 31/455; A61K 45/06

(52) U.S. Cl. CPC A61K 31/455; A61K 45/06

(57) ABSTRACT

In one embodiment, the present application discloses a method of reducing senescence in a mammal by reducing the concentration of non-ferritin iron within the mammal, comprising the administration of a therapeutically effective amount of an iron chelator or an antioxidant, or a pharmaceutically acceptable salt thereof.

### BACKGROUND

Like higher-order counterparts, Caenorhabditis elegans deposit fat, accumulate lipofuscin, develop sarcopenia and suffer neurodegeneration with age. The biochemical processes and underlying molecular mechanisms driving these events are uncertain. Insulin-like signaling (ILS) is a conserved pathway that has been shown to increase lifespan in nematodes, flies and mammals(1). Mutation in daf-2, encoding an insulin/IGF-1 receptor ortholog (2), doubles C. elegans life span but requires the activity of daf-16 (3), which encodes a FOXO family transcription factor (4). Mutation of daf-16 suppresses the longevity gains of daf-2 mutants. However, these daf-2 longevity mutants still age.

Previously, we identified that C. elegans express high concentrations of iron in intestinal cells, which house the majority of metabolic processes in this animal (5, 6). Biological iron exists predominantly in either ferrous (Fe²⁺) or ferric (Fe³⁺) oxidation states, with the majority of bioavailable iron tightly bound by enzymes and storage proteins. Exchangeable stores of iron are essential for incorporation of iron into functional metalloenzymes and heme groups, and are regulated by related storage (e.g. ferritin) and homeostatic proteins.

In one embodiment, in order to expand our understanding of the chemistry of aging for developing a therapeutic method for arresting or slowing the process of aging, we employed a population study of biological iron in whole C. elegans using synchrotron-based quantitative elemental imaging. In one aspect, a complimentary native-metalloproteomic analysis was developed to identify and understand how endogenous ligands of iron change during senescence.

The foregoing examples of the related art and limitations are intended to be illustrative and not exclusive. Other limitations of the related art will become apparent to those of skill in the art upon a reading of the specification and a study of the drawings or figures as provided herein.

### SUMMARY

Iron is essential for eukaryotic biochemistry. Extensive trafficking and storage of iron is required to maintain supply while preventing it from generating radicals and reactive oxygen species (ROS). In one embodiment, the present application discloses a method of employing population level X-ray fluorescence imaging and native-metalloproteomic analysis to determine and establish that iron accumulation is a pathological determinant of aging in a mammal.

In one aspect, the present application discloses the mechanism and location where iron homeostasis is lost during senescence, and its relationship to the age-related elevation of damaging reactive oxygen species. The application identifies both the genetic and drug-based interventions that target iron homeostasis to extend lifespan of a mammal. In one aspect, the lifespan is extended by at least 10%, 20%, 30%, 40%, 50%, 60% or 70%. In another aspect, the present application discloses that the loss of iron homeostasis may be a fundamental and inescapable cause of aging.

In one embodiment, the present application discloses that iron accumulation underlies normal biological aging. In one aspect, the application defines the nature and source of this deleterious iron by using micro-scale direct imaging and mass spectrometry. Further disclosed herein is the first direct evidence for a mechanism for the increase of reactive oxygen species production mediated by iron dyshomeostasis, a critical piece of data that has eluded gerontology to date. Although the idea that this may occur has been postulated by the free radical theory of aging, this has not been tested so directly or definitively. As disclosed herein, deleterious iron can be reduced by genetic and pharmacological intervention to increase life span. In one embodiment, the present method provides a clear mechanism for understanding how loss of cellular iron homeostasis contributes to major age-related human diseases.

We employ population level X-ray fluorescence imaging and native-metalloproteomic analysis to determine that iron accumulation is a pathological determinant of aging in Caenorhabditis elegans. Wild types utilize ferritin to sustain longevity, buffering against exogenous iron and showing rapid aging if ferritin is ablated. After reproduction, iron escapes from safe-storage in ferritin in the aging C. elegans, enters cell nuclei and generates reactive oxygen species. However, deleterious iron accumulation can be attenuated both by known longevity mutations and by direct pharmacological intervention to markedly extend lifespan. These findings support the importance of iron-mediated processes that drive the mechanism of senescence.

In addition to the exemplary embodiments, aspects and variations described above, further embodiments, aspects and variations will become apparent by reference to the drawings and figures and by examination of the following descriptions.

### DESCRIPTION OF THE FIGURES

FIG. 1. Iron accumulation in aging C. elegans. FIG. 1(A) Representative XFM images highlighting the age related accumulation of iron in wild type adult C. elegans (4 and 12-day old), panels i & iii and ii & iv respectively). Inelastic scatter (Compton scatter) from the specimen (grey scale image) provides visualization of internal anatomy, overlaid in red is the distribution of iron where present at >5 fg μm⁻². Scale bar=100 μm. FIG. 1 (B) Quantitation of iron by XFM in individual animals at intervals over their lifespan. Overlaid are mean±SEM. ** denotes p<0.01 and *** p<0.001. Long-lived daf-2 mutants accumulate significantly less iron during aging (p<0.001). FIG. 1 (C) Bulk iron levels measured by ICP-MS from aging cohorts of wild type (shown is mean total iron per worm±SEM, 100 animals per measure). FIG. 1 (D) Bulk iron levels (measured via ICP-MS) from aging cohorts of wild type (shown is mean μmol iron per dry weight±SEM). FIG. 1 (E) Bulk iron levels from aging cohorts of daf-2 mutants (shown is mean μmol iron per dry weight±SEM). FIG. 1 (F) Histological staining for iron (brown) in young (4-day, left) and old (12-day, right) C. elegans. Top row: Head sections. Middle row: Mid-body sections. Bottom row: Posterior sections. Insets show i, intestinal cell nuclei free of iron in young C. elegans (filled triangles), ii, iron deposits close to the intestinal lumen in young adults, iii, iron in inclusions (box=close-up) within the head of old worms (filled arrowhead), iv, intranuclear iron within the intestine in old worms (open triangles).

FIG. 2. ROS generation is a product of iron accumulation. FIG. 2(A) Live imaging of exchangeable iron using Calcein-AM fluorescence in young (4-day, top) and old (12-day, bottom) C. elegans. Fluorescence is quenched by increased iron in the intestine of old worms. Bright field image (above) and fluorescence image (below) with an outline in yellow. FIG. 2 (8) Quantitation of in vivo ROS increases with age in whole wild type C. elegans (mean±SEM, n=9, 7 and 5 individuals worms respectively, *** p<0.001). FIG. 2(C) Ex vivo ROS generation is a product of iron accumulation. Long-lived daf-2 mutants lack the increase in levels of ROS (DCF fluorescence) with aging compared to wild type and daf-16;daf-2 mutants (mean±SEM, n=4, *** p<0.001).

FIG. 3. Redistribution of iron with aging. FIG. 3 (A) Typical iron levels in size exclusion chromatography fractions from lysate soluble fractions of C. elegans at different ages (as indicated). With aging, decreased iron is associated with ferritin (Peak #2) and increased iron in HMW (Peak #1) and LMW (Peak #3). FIG. 3(B) Quantitation of iron in the three major chromatographic peaks across age (mean±se, n=3, one-way ANOVA with Dunnett's post hoc test, **p<0.01, ***p<0.001,). Shown is a linear regression (dotted line) with corresponding coefficients of determination (R²). FIG. 3 (C) Loss of ftn-2 removes the iron-ferritin peak in young (5-day old) adults. FIG. 3 (D) Calcium and iron maps for 5-day old wild type (i) and null mutants for ftn-1 (ii) and ftn-2 (iii), imaged by 2-D XFM elemental mapping (images are typical of n=3). The graph represents the quantitation of iron per worm from the 2-D XFM data, mean±SEM, n=3, **, p<0.01.

FIG. 4. Genetic and pharmacological interventions to limit iron accumulation extend lifespan. FIG. 4 (A) LC-ICP-MS profiles of long-lived daf-2 mutants lack the age-dependent increases in iron in Peaks #1 and #3 compared to wild type and daf-16;daf-2 mutants. Representative data from duplicate experiments are shown. FIG. 4 (B) Safe storage of iron in ferritin is required for normal ageing and lifespan (median lifespan in days at 25° C.: wild type 18 days, n=72; ftn-2(−) 16 days, n=71, p<0.001; ftn-2(−); ftn-1(−)15 days, n=71, p<0.001) FIG. 4 (C) Iron scavenging by SIH significantly extends wild type C. elegans lifespan (median lifespan at 25° C.: 0 μM SIH 12 days, n=91; 100 μM SIH 17 days, n=100, p<0.001; 250 μM SIH 21 days, n=103, p<0.001). Lifespan data shown is representative of duplicate experiments. FIG. 4 (D) LC-ICP-MS profiles of 4 and 10 day old wild type treated without (CTL) and with 250 μM SIH (SIH). FIG. 4 (E) Integration of peak area from triplicate data shows SIH reduced ferritin-iron (***p<0.001) and Peak #3 (LMW-iron, *p<0.05).

FIG. 5. (A) Histological staining for Fe in young (4-day, top), post-reproductive (8-day, middle) and old (12-day, bottom) C. elegans. Mid-body sections distal gonad nuclei free of iron in young adults (solid triangles). Aged individuals have increasing ectopic iron deposits within the germline nuclei (open triangles). FIG. 5 (B) Total Cu per dry weight of sample increases with aged in wild type C. elegans. ANOVA F(2,14)=17.337, p<0.001, post-hoc (Tukey) tests where * denotes p<0.05). FIG. 5(C) Total Cu per dry weight of sample increases with aged in daf-2(e1370) C. elegans (** p<0.05, 2-tailed t-test). FIG. 5 D, Total calcium increases with age in wild type (p<0.05, 2-tailed t-test). Shown are mean±SEM from age matched individual XFM images.

FIG. 6. (A) In vivo ROS production detected by DCF fluorescence in the intestine of young (top) compared to old (bottom) C. elegans. As with iron, most of the ROS signal also comes from the intestine. FIG. 6 (B) Spectral analysis of DCF fluorescence following 485 nm excitation showing peak fluorescence at 528 nm. FIG. 6 (C) ROS production detected by DCF fluorescence from lysates of young adult wild type C. elegans (10 μg), including a positive control of 10 nM FeCl₃.EDTA. ROS production was silenced by addition of 50 μM diethylenetriamine penta-acetic acid (DTPA). FIG. 6 (D) DCF fluorescence increasing over time in lysates from 4, 8, 12 and 16-day old wild type, daf-2(−) and daf-16(−);daf-2(−) animals.

FIG. 7. Purification of native C. elegans ferritin. FIG. 7(A) Since Peak #2 is the major iron fraction at all ages, to identify the main protein component of this peak a large number of animals of mixed ages were pooled. 10 g of soluble fraction of C. elegans lysate was applied to isoelectric focusing (pH 3-10) and separated fractions were measured for iron content by atomic absorption spectroscopy. Fractions 6-8 were pooled and refocused. FIG. 7 (B) Fractions 4-9 were collected from a narrower isoelectric refocusing (pH 5-7). FIG. 7 (C) Native size exclusion of pooled samples resolved a iron containing peak. Fractions 9 and 10 (F9/10) were pooled for further analysis via immunoblotting and mass spectrometry. FIG. 7 (D) Oriole stained SDS-PAGE of 1) Horse ferritin, 2) Protein size standards and 3) F9/10, showing a protein species in F9/10 resolving at −19 kD. FIG. 7 (E) LC-ICP-MS of lysate from wild type C. elegans (starting material), F9/10 and Horse ferritin showing co-elution of native horse ferritin and material in F9/10. FIG. 7 (F) MALDI MS of identifies a single parent species of 19505.2 Da in F9/10 purified C. elegans FTN-2. FIG. 7 (G) MALDI-MS/MS of lysC-digested F9/10 identifies protein as FTN-2 (62% sequence coverage over 6 fragments, Mascot score of 136, p=2.3×10-5). Shown in bold are the residues in the identified fragments of FTN-2 identified residues. Note the starting methionine has been cleaved and the mature protein has a N-terminal acetylated.

FIG. 8. (A) Insoluble iron increases in ageing wild type C. elegans. Shown are bulk iron measures of ⁵⁶Fe per unit dry weight of the TBS insoluble fraction from aged cohorts of wild type (mean from triplicate measures±SEM). Histological staining for iron (brown) in wild type (above) FIG. 8(B, C) and ftn-2(−);ftn-1(−) null adult (5-day-old) FIG. 8(D, E). ftn-2(−);ftn-1(−) null animals have markedly reduced iron staining in the intestine. Shown are representative longitudinal and traverse cross sections. FIG. 8 (F) DNA Sequence analysis of ftn-1 null allele. Shown are intronic and untranslated sequences in lower case and exon sequence in (blue) upper case. The ftn-1(ok3625) lesion is underlined. FIG. 8 (G) Schematic of the ftn-1(ok3625) deletion and FIG. 8(H) predicted 20 amino acid truncation product from the ok3625 allele.

### DETAILED DESCRIPTION

### Definitions

Unless specifically noted otherwise herein, the definitions of the terms used are standard definitions used in the art of organic chemistry and pharmaceutical sciences. Exemplary embodiments, aspects and variations are illustrative in the figures and drawings, and it is intended that the embodiments, aspects and variations, and the figures and drawings disclosed herein are to be considered illustrative and not limiting.

“Pharmaceutically acceptable salts” means salt compositions that is generally considered to have the desired pharmacological activity, is considered to be safe, non-toxic and is acceptable for veterinary and human pharmaceutical applications. Such salts include acid addition salts formed with inorganic acids such as hydrochloric acid, hydrobromic acid, sulfuric acid, phosphoric acid, and the like; or with organic acids such as acetic acid, propionic acid, hexanoic acid, malonic acid, succinic acid, malic acid, citric acid, gluconic acid, salicylic acid and the like.

“Therapeutically effective amount” means a drug amount that elicits any of the biological effects listed in the specification.

While particular embodiments are shown and described herein, it will be obvious to those skilled in the art that such embodiments are provided by way of example only. Numerous variations, changes, and substitutions will now occur to those skilled in the art. It should be understood that various alternatives to the embodiments described herein may be employed in practicing the methods described herein. It is intended that the appended claims define the scope of the invention and that methods and structures within the scope of these claims and their equivalents be covered thereby.

Unless defined otherwise, all technical and scientific terms used herein have the same meaning as is commonly understood by one of skill in the art. All patents and publications referred to herein are incorporated by reference.

As used in the specification and claims, the singular form “a”, “an” and “the” include plural references unless the context clearly dictates otherwise.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

The terms “treatment,” “treating,” “palliating,” and “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal so that both agents and/or their metabolites are present in the animal at the same time. Co-administration includes simultaneous administration in separate compositions, administration at different times in separate compositions, or administration in a composition in which both agents are present.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions described herein is contemplated. Supplementary active ingredients can also be incorporated into the compositions.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. While preferred antagonists herein specifically interact with (e.g. bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor, or an undesired immune response as manifested in autoimmune disease.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g. bind to) the target, compounds that initiate or enhance a biological activity of the target polypeptide by interacting with other members of the signal transduction pathway of which the target polypeptide is a member are also specifically included within this definition.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize the limits to the structural nature of the agents described herein.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

The term “cell proliferation” refers to a phenomenon by which the cell number has changed as a result of division. This term also encompasses cell growth by which the cell morphology has changed (e.g., increased in size) consistent with a proliferative signal.

The term “selective inhibition” or “selectively inhibit” as applied to a biologically active agent refers to the agent's ability to selectively reduce the target signaling activity as compared to off-target signaling activity, via direct or interact interaction with the target.

“Subject” refers to an animal, such as a mammal, for example a human. The methods described herein can be useful in both human therapeutics and veterinary applications. In some embodiments, the patient is a mammal, and in some embodiments, the patient is human.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

Unless otherwise stated, structures depicted herein are also meant to include compounds which differ only in the presence of one or more isotopically enriched atoms. For example, compounds as described herein wherein hydrogen is replaced by deuterium or tritium, or the replacement of a carbon by ¹³C- or ¹⁴C-enriched carbon.

The compounds described herein may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds described herein, whether radioactive or not, are encompassed.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(..+−..)” is used to designate a racemic mixture where appropriate. “Diastereoisomers” are stereoisomers that have at least two asymmetric atoms, but which are not mirror-images of each other. The absolute stereochemistry is specified according to the Cahn-lngold-Prelog R—S system. When a compound is a pure enantiomer the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined. When the compounds described herein contain olefinic double bonds or other centers of geometric asymmetry, and unless specified otherwise, it is intended that the compounds include both E and Z geometric isomers.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Compounds described herein also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

“Solvent,” “organic solvent,” and “inert solvent” each means a solvent inert under the conditions of the reaction being described in conjunction therewith including, for example, benzene, toluene, acetonitrile, tetrahydrofuran (“THF”), dimethylformamide (“DMF”), chloroform, methylene chloride (or dichloromethane), diethyl ether, methanol, N-methylpyrrolidone (“NMP”), pyridine and the like. Unless specified to the contrary, the solvents used in the reactions described herein are inert organic solvents. Unless specified to the contrary, for each gram of the limiting reagent, one cc (or mL) of solvent constitutes a volume equivalent.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can also be used.

When desired, the (R)- and (S)-isomers of the compounds described herein, if present, may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

The compounds described herein can be optionally contacted with a pharmaceutically acceptable acid to form the corresponding acid addition salts. Pharmaceutically acceptable forms of the compounds recited herein include pharmaceutically acceptable salts, chelates, non-covalent complexes, prodrugs, and mixtures thereof. In certain embodiments, the compounds described herein are in the form of pharmaceutically acceptable salts. In addition, if the compound described herein is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) include those embodiments, for example, an embodiment of any composition of matter, composition, method, or process, or the like, that “consist of” or “consist essentially of” the described features.

Suitable iron chelators for use in the compositions and methods described herein include known iron chelators and isomers and derivatives thereof. Exemplary iron chelators include\, but are not limited to, siderophores such as deferoxamine and desferrithiocin ([2-(3-hydroxypiridin-2-yl)-4-methyl-4,5-dihydrothiazole-4-carboxylic acid); synthetic chelators (including acylhydrazones) such as salicylaldehyde isonicotinoyl hydrazone, deferiprone (Ferriprox®), clioquinol, 0-trensox (Tris-N-(2-aminoethyl-[8-hydroxiquinolie-5-sulfonato-7-carboxamido]amine), Deferasirox (ICL670, Exjade®); Tachpyr (N,N,N″-tris(2-pyridylmethyl)-cis,cis-1,3,5-triamino cyclohexane), Decrazone (ICRF-197), Triapine (3-aminopyridine-2-carboxaldehyde thiosemicarbazone), Pyridoxal isonicotinoyl hydrazine, and Di-2-pyiridylketone thiosemicarbazone; phytochemicals such as flavan-3-ol, curcumin, apocymin, kolaviron, floranol, baicelein, baicalin, ligustrazine (Lifusticum wallichi Francha), quercetin, epigallocatechin gallate, theaflavin, phytic acid, and genistein (5,7,4′-trihydroxyisoflavone).

The subject pharmaceutical compositions are typically formulated to provide a therapeutically effective amount of an iron chelator as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, an iron chelator and other agent(s) may be mixed into a preparation or both components may be formulated into separate preparations to use them in combination separately or at the same time.

In some embodiments, the concentration of one or more of the iron chelators in the pharmaceutical compositions described herein is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more of the iron chelators is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more of the iron chelators is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v.

In some embodiments, the concentration of one or more of the iron chelators is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more of the iron chelators is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the iron chelators is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the iron chelators is in the range of 0.0001-10 g, 0.0005-9 g, 0.001-8 g, 0.005-7 g, 0.01-6 g, 0.05-5 g, 0.1-4 g, 0.5-4 g, or 1-3 g.

The iron chelators described herein are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the iron chelator is administered, the subject to be treated, the body weight of the subject to be treated, and the preference and experience of the attending physician.

A pharmaceutical composition described herein typically contains an active ingredient (e.g., an iron chelator or a pharmaceutically acceptable salt and/or coordination complex thereof, and one or more pharmaceutically acceptable excipients, carriers, including but not limited inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration

Described herein is a pharmaceutical composition for oral administration containing an iron chelator, and a pharmaceutical excipient suitable for oral administration.

Also described herein is a solid pharmaceutical composition for oral administration containing: (i) an effective amount of an iron chelator; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

Also described herein are anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. Pharmaceutical compositions and dosage forms which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions described herein to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound described herein and to minimize precipitation of the compound described herein. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, .delta.-valerolactone and isomers thereof, .beta.-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

Described herein are pharmaceutical compositions for injection containing an iron chelator and a pharmaceutical excipient suitable for injection. Components and amounts of agents in the compositions are as described herein.

The forms in which the novel compositions described herein may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating an iron chelator in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery.

Also described herein is a pharmaceutical composition for transdermal delivery containing an iron chelator and a pharmaceutical excipient suitable for transdermal delivery.

Compositions described herein can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods described herein employs transdermal delivery devices (“patches”). Such transdermal patches may be used to provide continuous or discontinuous infusion of an iron chelator in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the iron chelators or pharmaceutical compositions described herein can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of an iron chelator administered will be dependent on the mammal being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, an iron chelator is administered in a single dose. Typically, such administration will be by injection, e.g., intravenous injection, in order to introduce the agent quickly. However, other routes may be used as appropriate.

In some embodiments, an iron chelator is administered in multiple doses. Dosing may be about once, twice, three times, four times, five times, six times, or more than six times per day. Dosing may be about once a month, once every two weeks, once a week, or once every other day. In another embodiment a compound and another agent are administered together about once per day to about 6 times per day. In another embodiment the administration of an iron chelator and an agent continues for less than about 7 days. In yet another embodiment the administration continues for more than about 6, 10, 14, 28 days, two months, six months, or one year. In some cases, continuous dosing is achieved and maintained as long as necessary.

Administration of the iron chelator(s) may continue as long as necessary. In some embodiments, an iron chelator is administered for more than 1, 2, 3, 4, 5, 6, 7, 14, or 28 days. In some embodiments, an iron chelator is administered for less than 28, 14, 7, 6, 5, 4, 3, 2, or 1 day. In some embodiments, an iron chelator is administered chronically on an ongoing basis, e.g., for the treatment of chronic effects.

An effective amount of an iron chelator may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions described herein may also be delivered via an impregnated or coated device such as a stent, for example, or an artery-inserted cylindrical polymer. An iron chelator may be administered, for example, by local delivery from the struts of a stent, from a stent graft, from grafts, or from the cover or sheath of a stent. In some embodiments, an iron chelator is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. An iron chelator may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, an iron chelator may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of an iron chelator in a suitable solvent, followed by evaporation of the solvent. Excess drug on the surface of the stent may be removed via an additional brief solvent wash. In yet other embodiments, an iron chelator may be covalently linked to a stent or graft. A covalent linker may be used which degrades in vivo, leading to the release of an iron chelator. Any bio-labile linkage may be used for such a purpose, such as ester, amide or anhydride linkages. An iron chelator may additionally be administered intravascularly from a balloon used during angioplasty. Extravascular administration of an iron chelator via the pericard or via advential application of formulations described herein may also be performed to decrease restenosis.

A variety of stent devices which may be used as described are disclosed, for example, in the following references, all of which are hereby incorporated by reference: U.S. Pat. No. 5,451,233; U.S. Pat. No. 5,040,548; U.S. Pat. No. 5,061,273; U.S. Pat. No. 5,496,346; U.S. Pat. No. 5,292,331; U.S. Pat. No. 5,674,278; U.S. Pat. No. 3,657,744; U.S. Pat. No. 4,739,762; U.S. Pat. No. 5,195,984; U.S. Pat. No. 5,292,331; U.S. Pat. No. 5,674,278; U.S. Pat. No. 5,879,382; U.S. Pat. No. 6,344,053.

The iron chelators may be administered in dosages. It is known in the art that due to intersubject variability in compound pharmacokinetics, individualization of dosing regimen is necessary for optimal therapy. Dosing for an iron chelator may be found by routine experimentation in light of the instant disclosure.

When an iron chelator, is administered in a composition that comprises one or more agents, and the agent has a shorter half-life than the iron chelator unit dose forms of the agent and the iron chelator may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and an iron chelator as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

Kits are also described herein. The kits include one or more iron chelators as described herein, in suitable packaging, and written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, an iron chelator and the agent are provided as separate compositions in separate containers within the kit. In some embodiments, the compound described herein and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### EXAMPLES

### Materials and Methods

Strains.

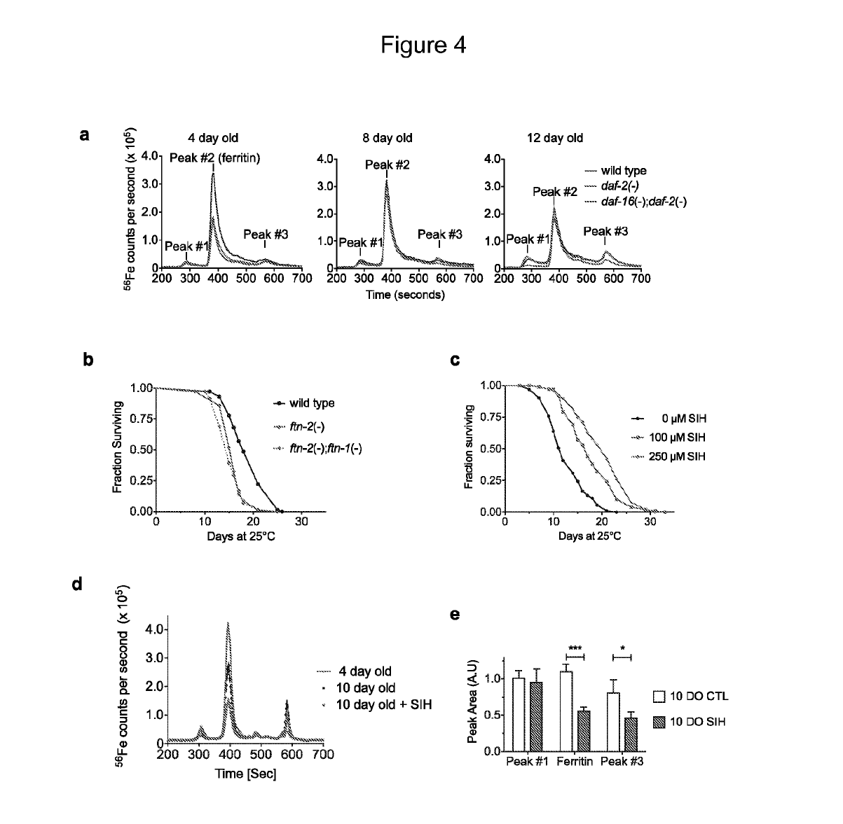

N2 (wild-type), TJ1060: spe-9 (hc88); fer-15 (b26), CB1370: daf-2 (e1370), and DR1309: daf-16 (m26); daf-2 (e1370), RB2603; ftn-1(ok3625) and RB668; ftn-2 (ok404) were obtained from the Caenorhabditis Genetics Center. As the ftn-1(ok3625) deletion allele has not been mapped, we sequenced the genomic DNA across the putative deletion site. A 495 bp deletion was identified that removes all of exon 2 and most of exon 3 (FIG. 4 f-h), resulting in a premature stop codon (UGA), so that the likely product is truncated to 20 amino acids (out of the predicted 170). The ftn-2(ok404) is a previously-characterized null allele(14). RB2603; ftn-1(ok3625) and RB668; ftn-2(ok404) were each backcrossed four times to wild type prior to further analysis. All strains were maintained at 20° C. on standard nematode growth media (NGM) (28) and aged at 25° C. as required, with the exception of the fertility mutant TJ1060 which was maintained at 16° C.

X-Ray Fluorescence Microscopy (XFM).

Aged C. elegans were prepared as previously described (6) on Si₃N₄ windows (Silson) for analysis at the XFM beamline at the Australian Synchrotron and the experimental setup has been described previously (5). The distribution of metals was mapped using a beam of 12.7 keV X-rays focused to 2 μm (full-width at half-maximum of the intensity) using a Kirkpatrick-Baez mirror pair(29). The X-ray energy was chosen to induce K-shell ionization of elements with atomic numbers below 34, while also separating elastic and inelastic scatter from the fluorescence of lighter elements. The specimen was continuously scanned through the X-ray focus using a step size of 2 μm. Entire X-ray fluorescence (XRF) spectra were obtained using an effective dwell time of −8 ms per pixel. XRF was recorded using the low-latency, large solid angle 384-channel Maia XRF detector (30). Resulting elemental maps ranged up to 50,000 pixels in size and the total acquisition time varied around 7 minutes per specimen. Two single-element foils of known areal density, Mn and Pt (Micromatter, Canada), were scanned during the experiment as references for determination of elemental areal density (31).

Comparison of total iron quantitated from whole worms was examined via one-way ANOVAs with a Dunnett's post-hoc test. Within each genotype aged cohorts were compared to young (4 days of age) adults.

Pooled values from age-matched wild type C. elegans and ferritin mutant XFM images were used to generate histograms of iron concentration per pixel. Comparisons between the goodness of fit for nested models, i.e. Gaussian (X B N(m,s2)) vs. a sum of Gaussians (Pni1/41 Xi) with respect to measured data were assessed using the Akaike information criterion and the exact sum of squares F-test. The dependency and co-variance between parameters of a candidate model were monitored and if the model contained redundant parameters the model was rejected. Throughout this work the significance level is defined as p≦0.05 and graphical data are presented as the mean±SEM unless otherwise noted.

Histology.

C. elegans were washed in S-basal (28), fixed overnight in 10% (v/v) neutral buffered formalin (NBF) at 4° C., embedded in 2% (w/v) agar in phosphate buffered saline (PBS) blocks and then fixed again in 10% NBF overnight. Following processing of the agar blocks into paraffin, 5 μm sections were prepared, dewaxed and stained with DAB-enhanced modified Perl's Prussian blue following a standard protocol (32). Samples were counter stained with Harris haematoxylin solution (Amber Scientific).

Live Imaging of Iron.

C. elegans cultures were aged as indicated, washed in S-basal (28), then co-cultured in S-basal containing 1×10⁸ cells OP50 (E. coli) and 0.05 μg/ml Calcein-AM (Invitrogen) for 1 h and then in S-basal with 1×10⁸ cells OP50 for 1 h. Samples were then mounted for epi-fluorescence microscopy using standard techniques. Calcein fluoresces in the presence of calcium ions in solution, but this fluorescence is quenched by ionic iron. Calcein has a slight selectivity for Fe′ over Fe′. Other divalent metal ions, Cu²⁺, Ni²⁺ and Co²⁺ can also quench Calcein fluorescence, however, these were found to be present at >2 orders of magnitude lower than Fe in the intestine (FIG. S2) and therefore not considered to be able to interfere with the signal.

ROS Detection.

2′,7′-dichlorodihydrofluorescein diacetate (DCFH-DA) at 10 μM was used as a fluorescent probe for reactive oxygen species (ROS) detection in vivo using standard protocols (33). DCFH-DA enters and accumulates in the intestinal cells, where it is oxidized by several ROS (including hydroxyl radicals) to form the fluorophore DCF(34). Analysis of fluorescence rate increase was performed on samples sonicated in 1×TBS, pH 7.4 and recovered as the supernatant from a 100,000 g centrifugation at 4° C. Total protein concentration was determined by a Nanodrop spectrophotometer (Thermo Scientific). Lysate supernatants (50 μg total protein) were brought to a reaction volume of 200 μl with 200 mM ammonium acetate pH 7.5, 100 μM DCFH-DA (Sigma-Aldrich, made from a 10 mM stock in acetonitrile), and 400 μM ascorbate, in black 96-well micro titer plates. Fluorescence in 8 replicate wells was quantitated (Eₓ: 485 nm, Eₘ: 535 nm using a 495 nm cut-off) by a FlexStation (Molecular Devices) plate reader, using 30 reads every minute for 1 h at medium PMT setting. Values from lysate-free (negative) controls were subtracted and the fluorescence data was then base line corrected. A linear regression fitted from 20-40 minutes and the slopes were normalized against 4-day old wild type and plotted (Prism v5.0d, Graphpad Software).

Fluorescence Microscopy.

Animals were mounted on a glass slide with 2% (w/v) agar pad containing 2% NaN₃ under a glass cover slip and examined using an Olympus BX40 epifluorescence microscope equipped with SPOT RTKE cooled color CCD camera (Diagnostic Instruments, MI). The GFP fusion protein was visualized by using an Olympus U-MWG filter set (Eₓ: bandpass 530±20 nm, Eₘ: longpass 590 nm), and the images imported into SPOT software (Diagnostic Instruments). ImageJ v1.45s (NIH, USA) was used for image preparation and overlays.

Bulk Iron Quantitation.

Total iron was measured using a 7700 series (Agilent) inductively coupled plasma mass spectrometry (ICP-MS) as previously reported(27). Samples consisted of 100 adults per replicate for different aged cohorts as indicated.

Purification of C. elegans Ferritin.

10 g of mixed stage wild type cultured on 8P media (35) at 20° C. were frozen as pellets in liq-N2. Pellets were crushed in a liq-N2 chilled mortar and pestle, then added to 20 ml PBS pH 7.4 with EDTA-free proteinase inhibitors (Roche). The lysate was further disrupted with 20 strokes of an ice-cooled Dounce homogenizer (in 50 mL). The extract was centrifuged at 3300 g at 4° C. and the supernatant (˜30 mL) dialyzed overnight in 18 MΩ pure H₂O (Millipore) at 4° C. using pleated dialysis tubing with a 10 kDa molecular weight cut-off (Thermo Scientific). 20 ml of dialysate was then diluted to 60 ml with pH 3-10 ampholytes and iso-electrically focused via a Rotofor II (Bio-Rad), as per manufacturer's protocols to 2500 Vh. Fractions were collected and iron content measured by graphite furnace atomic absorption spectroscopy (AAS). A standard method was used with ashing and atomisation temperatures of 700° C. and 2300° C. respectively, and with linear absorbance to a concentration of 100 μg/L. The three highest contiguous iron-containing fractions were then pooled and re-focused via the Rotofor II as above. Fractions containing iron were identified by AAS. To the six contiguous iron containing fractions NaCl was added to a final concentration of 150 mM and then size-excluded via FPLC (Bio-Rad) using a Superdex 200 10/300 GL column and PBS buffer at 0.6 ml/min. Fractions were collected and iron was measured by AAS. Fractions 9 and 10 (F9/10) were identified, pooled and concentrated to 300 μl via vacuum centrifugation (SpeedVac, Savant). Aliquots were then frozen at −80° C. until required for further analysis.

Electrophoretic Analysis.

Samples were suspended in 1× Laemmli sample buffer (with 10 mM TCEP, 6 M urea, and 2% SDS), boiled for 10 min, and analyzed by SDS-PAGE (NuPage 4-12% Bis-Tris, Invitrogen). Samples were prepared in parallel were either stained with Oriole (Bio-Rad) in preparation for mass spectrometry or immunoblotted using a 1:1000 dilution of polyclonal anti-horse spleen ferritin antibody produced in rabbit (Sigma-Aldrich), and imaged via standard chemiluminescence.

Mass Spectrometry.

Matrix-assisted laser desorption/ionization-mass spectrometry (MALDI-MS) using an UltrafleXtreme (Bruker Daltronics), a two-layer sample preparation method (36) and α-cyano-4-hydroxycinnamic acid as the MALDI matrix, was used to determine the protein parent mass of the purified C. elegans F9/10 as ferritin. For further analysis the purified C. elegans ferritin was digested with LysC (Roche) in 4 M urea in a ratio of 50:1 protein to LysC overnight at 37° C. Digested material was then desalted and concentrated through a 04 ZipTip (Millipore) for MALDI-MS/MS analysis (as above) to generate a peptide mass fingerprint. The peptide masses were searched using MASCOT (Matrix Science).

DNA Sequencing.

The ftn-1 ORF amplicon was amplified using the following nested primers: Outer-forward 5′-ATGTGTCTCAGATTTCCGCC, Inner-forward 5′-GGTTGAACCTTTTTAGGAACTGC, Inner-reverse 5′-ACAGTCCCGGACACGTAATC and Outer-Reverse 5′-GAACCCTTTCGTTGCCAATA. Sequencing was performed using the inner primer pair at the Applied Genetic Diagnostic facility (Department of Pathology, University of Melbourne) using ABI3130xl capillary genetic analyzers and BDV3.1 terminators. Three independent ampicons, from both wild type and ftn-1(ok3625), were sequence on both complementary DNA strands.

Size Exclusion Chromatographic with Tandem Inductively-Coupled Plasma Mass Spectrometry (SEC-ICP-MS).

Samples were homogenized in TBS (pH 8.0) with added proteinase inhibitors (EDTA-free, Roche), then clarified by a 15 min centrifugation at 175,000 g, 4° C. The protein concentration of the supernatant was determined via UV absorbance (Nanodrop, ThermoScientific) and equivalent protein amounts were size-excluded using a Bio SEC-5 (4.6×300 mm, 5 μm, Agilent) column with 200 mM ammonium nitrate (trace analysis grade, Sigma) pH 8.0 buffer the flow rate was 0.4 mL/min at 30° C. The eluant from the column was directly connected to the ICP-MS for elemental detection as previously described (37).

Lifespan Analysis.

The effects of genetic ablation of ftn-2 and ftn-1 on wild type was measured using established protocols(11, 38). For SIH treatment compound was dissolved in dimethyl sulfoxide (Sigma-Aldrich) then added to the molten NGM at 55° C. along with 50 μg/mL amplicillin (Sigma-Aldrich). Media containing equivalent vehicle alone (0.5% v/v DMSO) and ampicillin was used for comparison. SIH data was collected using the temperature sensitive-sterile strain TJ1060. All life span assays were conducted at 25° C. following adulthood.

Statistical Tests.

Kaplan-Maier survival curves were generated and compared via non-parametric Log rank tests (Prism v5.0d, Graphpad Software).

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

### Example 1

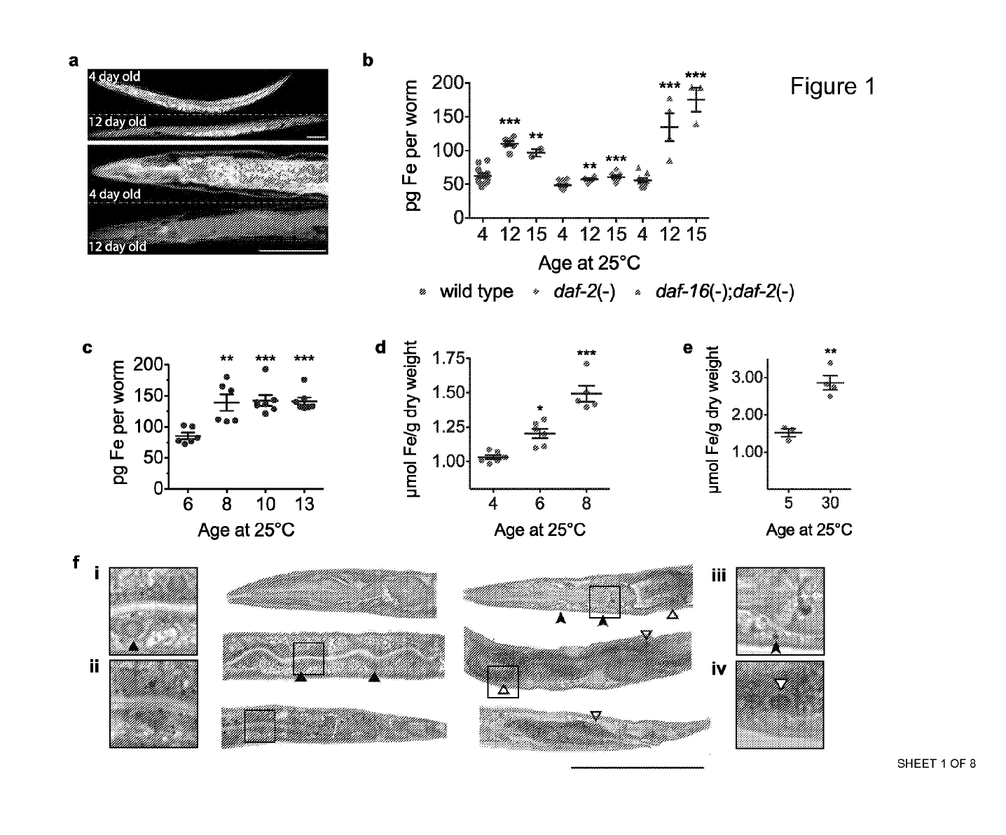

We examined the spatial distribution of iron in young and aged wild-type adult C. elegans (FIG. 1a) by quantitative whole-body X-ray fluorescence microscopy (XFM, ˜1 μm resolution) (6, 7). This revealed a 77% increase in mean total iron from young adults (4 days post egg lay, 62.5 μg iron per individual) to post-reproductive old animals (12 days old, 110.4 μg iron per individual; p<0.001, FIG. 1b), with marked intracellular accumulation in the intestinal cells (FIG. 1a). Bulk measures of iron using inductively coupled plasma-mass spectrometry (ICP-MS) in aging C. elegans cohorts (n=100 adults per aliquot) confirmed a significant increase in total iron (FIG. 1c) in post-reproductive senescent populations, e.g. 10-day old adults have a 66% increase in mean iron compared to 6-day old adults (p<0.001). Iron also increased with age when normalized against dry weight, indicating that the iron elevation is not due to increased body mass (FIG. 1d).

C. elegans canonical mutants of the ILS pathway that have altered rates of aging were also examined by XFM. The age-dependent rise in iron was markedly suppressed in the long-lived daf-2 mutants (FIG. 1b). Loss of daf-16 (which reverses the longevity effects of the daf-2 mutant) restored, and even exaggerated, the age-dependent rise in iron (FIG. 1b). When aged to their approximate median lifespan, daf-2 mutants also showed a marked increase in total iron, consistent with iron elevation heralding death even where the rate of aging is slowed (FIG. 1e).

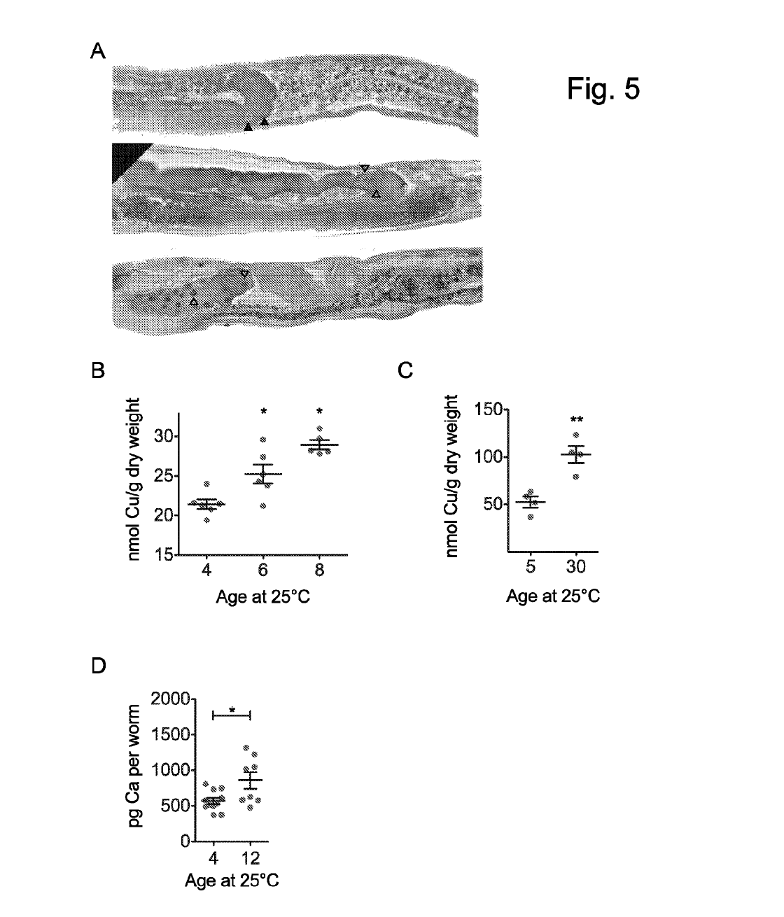

Young and old adult wild-type C. elegans were examined at higher resolution by histological Perls' staining for non-heme iron (8). In agreement with the XFM data, we found that iron accumulated in intestinal cells, progressing from discrete vesicular to dispersed distribution (FIG. 1f). Unlike 4-day-old C. elegans, 12-day-old worms developed iron staining within intestinal cell nuclei, and in inclusions in the head region. Adult C. elegans are post-mitotic and lose intestinal cell nuclei during aging by an unknown process (9), which our findings indicate is associated with toxic nuclear iron accumulation. In addition, we observed conspicuous iron accumulation in the germ nuclei of post-reproductive adults (FIG. 5a).

We hypothesized that the age-dependent elevation of iron we observed could generate deleterious redox activity (10) if the accumulation is not coordinated in a redox-silenced manner, for example, by ferritin. Copper levels also rose significantly with aging (FIG. 5b & c), and while this may also contribute to oxidative damage, the ˜100-fold greater abundance of iron compared to copper (11), indicating that the iron elevation is more likely to be problematic.

### Example 2

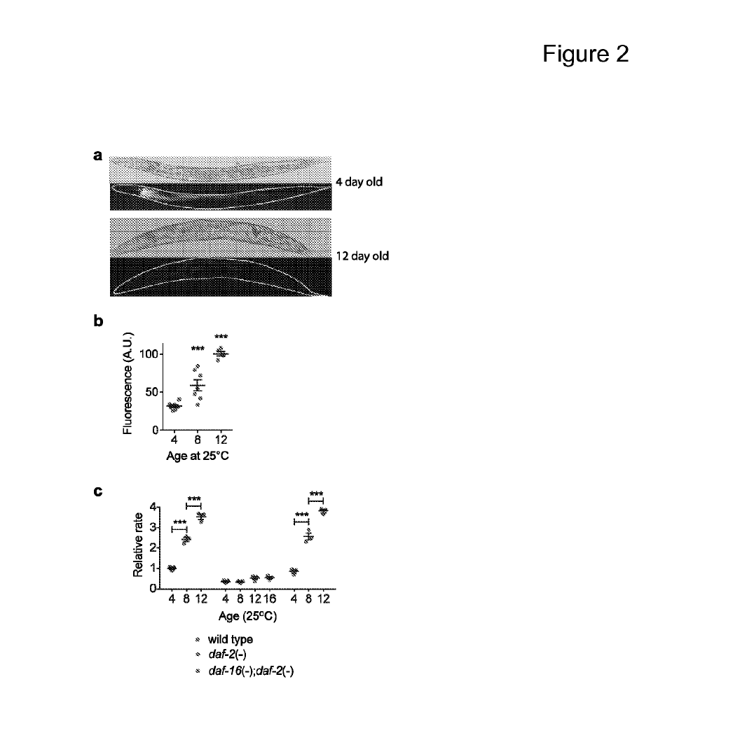

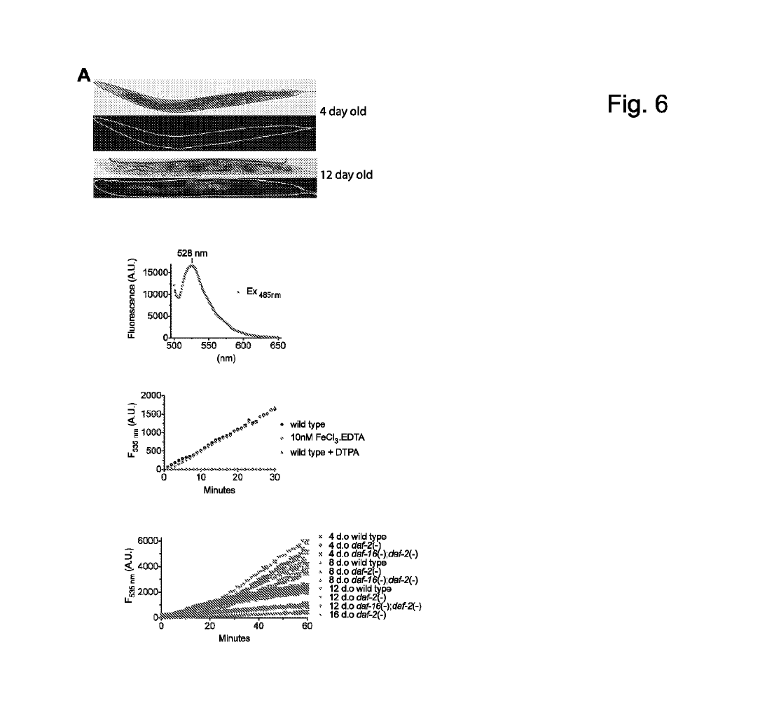

We investigated whether there is a relationship between reactive iron elevation and ROS generation within post-mitotic intestinal cells during aging in living C. elegans. Exchangeable iron was markedly elevated between day 4 and 12 in the intestine, as detected by suppression of calcein fluorescence (FIG. 2a) that could not be explained by a drop in calcium levels (FIG. 5d). Concomitantly, in vivo ROS (detected by 2′,7′-dichlorodihydrofluorescein diacetate, DCFH-DA, FIG. 6a) markedly increased in the intestine (p<0.001, one-way ANOVA d.f.=2, FIG. 2b).

To test whether age-dependent iron elevation is the catalytic source of the in vivo ROS elevation, we measured the initial rate of ROS production from the soluble fraction of lysed C. elegans, which we incubated ex vivo with DCFH-DA. In this assay, the rate of ROS generation is primarily proportional to the catalytically reactive pool of iron (with a small contribution possible from comparatively scarce redox-active metal ions copper and manganese). The rates of ex vivo ROS generation from wild-type C. elegans (FIG. 2c) rose throughout lifespan, matching the steady-state in vivo ROS values (FIG. 6a). Supporting the contribution of iron, ROS generation was abolished by the redox-silencing chelator diethylenetriamine penta-acetic acid, DTPA (FIG. 6c). Paralleling iron levels, the rates of ex vivo ROS production were significantly attenuated in the long-lived daf-2 mutants at each of the ages studied, remaining below the reference baseline rate (4 day old wild-type C. elegans) even by 16 days of age (FIG. 2d, FIG. 6d). As with its impact on iron levels with aging, daf-16 mutation restored age-related ROS elevation in the daf-16; daf-2 double mutants, consistent with the elevated ROS contributing to the abbreviated life-span.

### Example 3

When iron accumulates in a cell, it is normally sequestered by ferritin, which oxidizes Fe′ to hydrous ferric oxide in an exchangeable cytoplasmic reservoir protected from incidental redox reactions. Ferritin proteins are highly conserved and typically organize as a 24-mer capable of storing up to −4500 atoms of iron (although rarely saturated in vivo) (12).

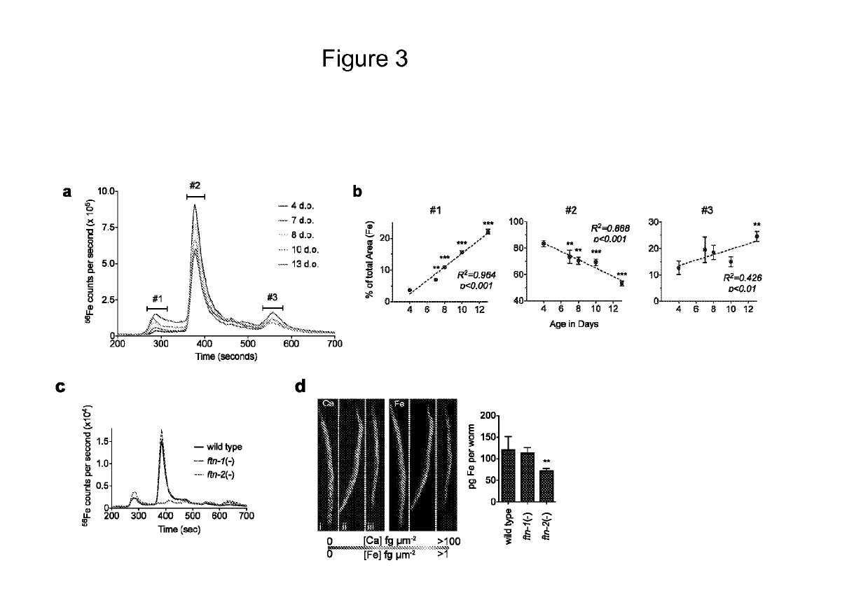

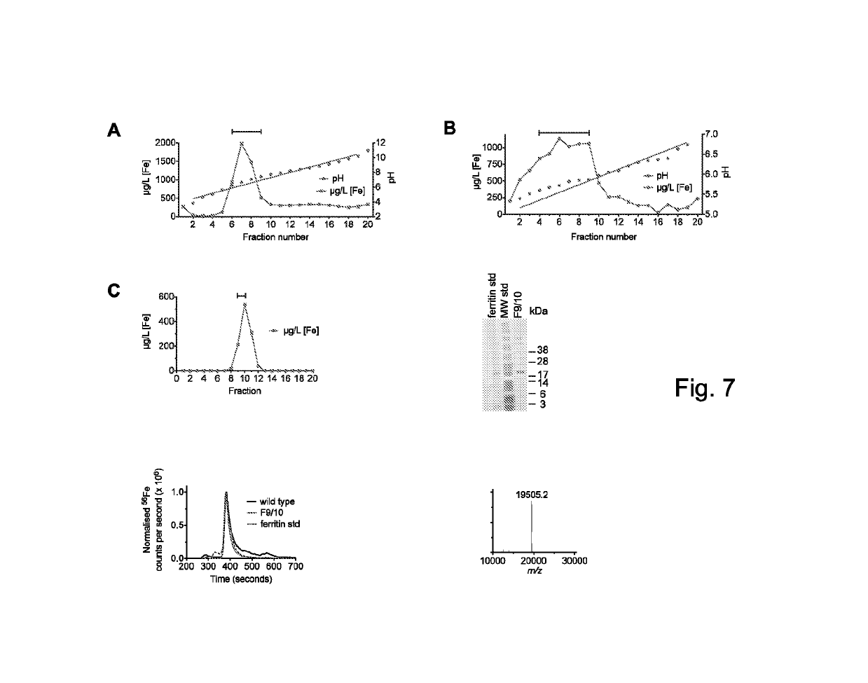

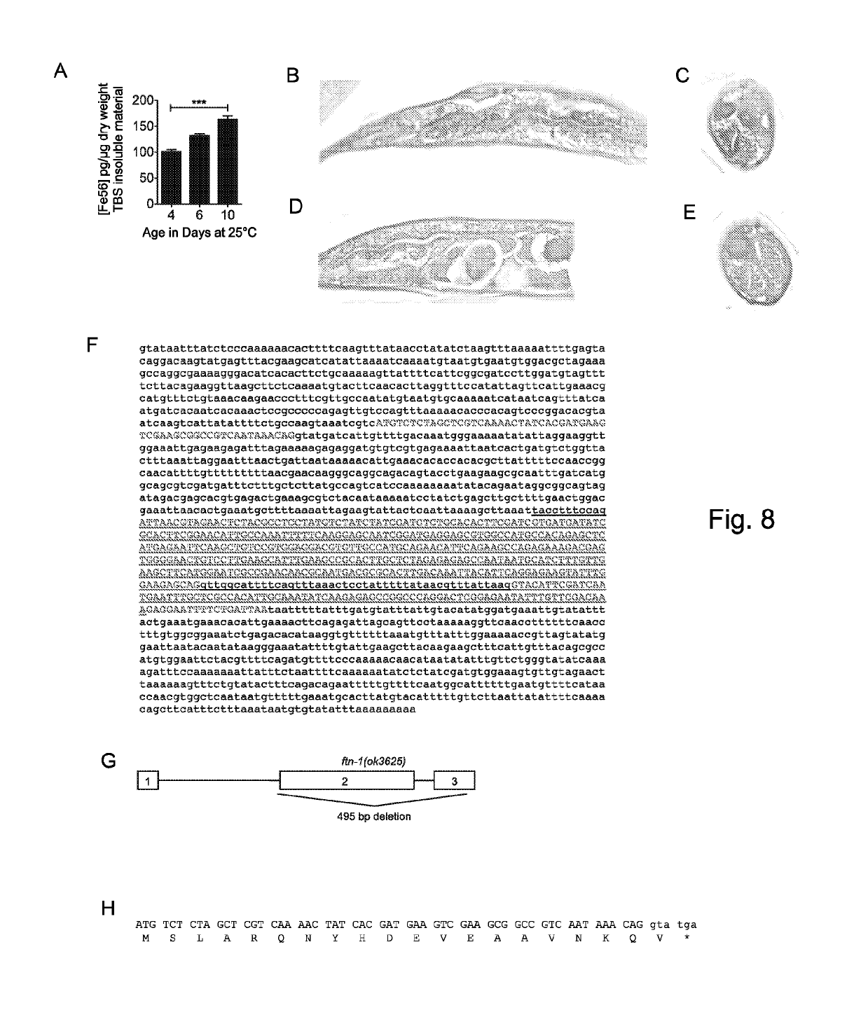

To determine whether the age-related tandem elevation of iron with ROS we observed was caused by loss of ferritin sequestration of iron, we analyzed iron in cytoplasmic biomolecules of homogenized aging C. elegans using native size-exclusion chromatography with on-line ICP-MS detection (SEC-ICP-MS). Soluble protein-bound iron from C. elegans resolved into three major peaks (FIG. 3a-b). Iron bound to Peak #2 represented most (˜84%) of the total soluble iron in 4-day-old wild-type worms. Peak #2 was identified as ferritin, FTN-2, by mass spectrometric analysis, FIG. 7). With aging (from 4 to 13 days, FIG. 3b), soluble iron redistributed away from ferritin (˜47%) and towards high molecular weight (HMW)-iron (>1 MDa, Peak #1, which increased 5-fold to become 20% of total soluble iron), and towards low molecular weight (LMW) iron species (<30 kDa, Peak #3, which increased ˜13% to become 25% of the total soluble iron pool). Integration of all peaks indicated that total soluble iron did not change with age, rather, the increased burden of iron we observed with aging (FIG. 1b & c) was driven by elevations in the insoluble fraction (˜60%, p<0.001, FIG. 8a).

### Example 4

The C. elegans genome encodes two heavy-chain ferritin orthologs, ftn-1 and ftn-2 (13). FTN-1 (predicted MW=19524.8) expression is induced in the intestine by high iron exposure, while FTN-2 (observed MW=19502.2) has constitutive as well as iron-responsive expression in intestinal cells (14, 15). Mass spectrometry detected no peptides of FTN-1 within the predominant iron-binding protein fraction. Therefore, aging is associated with the escape of iron from redox-protected storage in FTN-2 (FIG. 3b) to become species that foster ROS generation (FIG. 2b, c).

We investigated the ferritin-null C. elegans as young (5-day-old) adults. Genetic ablation of FTN-2 abolished chromatographic Peak #2 (ferritin-bound iron) and increased Peak #1, whereas loss of FTN-1 had only minor effects on the iron chromatogram (FIG. 3c) probably because of its low constitutive expression (15). Wild type and null mutants for ftn-1 and ftn-2 were imaged using XFM, which revealed that ftn-2 nulls had ˜46% less total iron within the intestinal cells (FIG. 3d), compared to wild type. Total iron in ftn-1 nulls was not significantly different from wild type. These data help interpret the age-dependent elevations in histological and total iron in wild type worms. Young ftn-1; ftn-2 null animals had no detectable iron staining (FIG. 8b-e), which indicates that the small amount of iron staining detected in young adult wild type C. elegans (FIG. 1f) is from ferritin. Iron staining markedly increases and spreads in the aging animal (FIG. 1f), but this must represent iron that fails to be incorporated into ferritin since we found that FTN-2 loading of iron decreases with age (FIG. 3a & b), consistent also with ferritin expression falling at the same time (16). Therefore, the age-dependent increase in histological iron we observed must reflect HMW (Peak #1), LMW (Peak #3) and/or insoluble accumulations.

We examined ILS pathway longevity mutants on the chromatographic distribution of iron upon aging. Total soluble iron was unaltered by these mutations in young (4-day-old) adults (FIG. 1b). Consistent with iron redistribution playing a pivotal role in lifespan, the daf-2 mutation, which increases lifespan approximately 70% (3), markedly attenuated the age-related increases in iron Peaks #1 and #3 seen in wild-type worms (FIG. 4a). This suppression of iron accumulation accounts for the low ROS generated by the daf-2 mutants (FIG. 2d). Conversely, loss of daf-16 (i.e. daf-16; daf-2 double mutants), which negates the lifespan increase caused by daf-2 mutation (3), restored both the prominent increases in Peaks #1 and #3 with age (FIG. 4a) and the tandem increase in ROS (FIG. 2d). The targets of DAF-16 (a FOXO transcription factor) have recently been implicated in iron homeostasis in C. elegans (17), and our data indicate that DAF-16 acts to prevent age-related toxic accumulation of iron.

Diminished ferritin storage of iron, which we found to be a feature of aging in C. elegans (FIG. 3a, b), could promote iron-mediated oxidative damage. Consistent with this, genetic ablation of ftn-2 alone or in combination asftn-2(−); ftn-1(−) significantly reduced lifespan (FIG. 4b). Conversely, we found that the long-lived daf-2 mutants do not store more iron in ferritin under normal conditions (FIG. 4a), despite being reported to express elevated basal ftn-1 mRNA (18).

### Example 5

To test whether morbid age-related iron accumulation can be therapeutically targeted, C. elegans were treated from adulthood onwards with salicylaldehyde isonicotinoyl hydrazone (SIH). This lipophilic compound belongs to class of acylhydrazones able to scavenge intracellular iron to facilitate extracellular clearance (19).

SIH treatment resulted in a robust 75% increase in lifespan (control versus 250 μM SIH median lifespan; 12 versus 21 days, p<0.001, FIG. 4c), and SIH also showed dose-dependency. In parallel, the chromatographic distribution of iron in C. elegans aged to 10 days (post adulthood) (FIG. 4d) revealed that SIH lowered LMW-iron (Peak#3) (normalized peak area −43%, p<0.05, FIG. 4e). Ferritin-bound iron (Peak #2) was also similarly decreased by SIH (p<0.01, FIG. 4e), but HMW-iron (Peak#1) was unaffected. In comparison, total soluble zinc was unaffected by SIH treatment (data not shown).

### DISCUSSION

Iron is an essential redox-active element for eukaryotes that can induce uncontrolled oxidative chemistry if left unchecked. We have applied several advanced analytical approaches to characterize iron accumulation as a potentially imperative feature of C. elegans senescence.

We hypothesize that the underlying mechanism of aging in wild type C. elegans is the escape of iron from safe storage in ferritin (FTN-2), where it emerges as redox-active species that redistribute primarily to soluble HMW-iron and insoluble precipitates where it catalyzes ROS generation. HMW-iron may represent misfolded, mis-metallated proteinaceous material transitioning towards an insoluble aggregate typical of aging pathology (20). The withdrawal of soluble iron from the cytoplasm into these aggregates could explain why the rise in total somatic iron is not sensed by the transcriptional mechanisms that should upregulate the expression of ferritin (13).

Our data indicate that the C. elegans intestine is particularly susceptible to senescent iron changes. Intestinal cell nuclei are progressively lost as C. elegans age (9), and the resulting cumulative loss of intestinal function is likely to further compound loss of iron homeostasis. We observed nuclear iron accumulation (FIG. 1f) within the intestine that is likely to contribute to demise of these nuclei. Intra-nuclear aggregates of ferritin have also been observed in mammalian models of toxic iron overload (21, 22).

The C. elegans genome encodes two ferritin genes; ftn-1 and ftn-2, however we have determined that only ftn-2 significantly contributes to iron storage under basal conditions. Although understanding the specific role ftn-1 function will require further investigation, our data are consistent with previous reports showing the impact of ftn-1 is negligible with respect to lifespan under normal conditions (23).

The age-dependent rise in iron is markedly delayed in long-lived daf-2 mutants. Yet, at their median lifespan, daf-2 mutants still show a significant increase in total iron (FIG. 1e), demonstrating that iron elevation invariably heralds death even where the rate of aging is slowed. The suppression of iron accumulation accounts for the low ROS generated by the daf-2 mutants. Loss of daf-16 by mutation reverses the longevity effects of the daf-2 mutant and restores and exaggerates the age-dependent rise in iron (FIG. 1b). DAF-16 need be expressed only in the intestine to slow aging in C. elegans (24). This is consistent with uncontrolled iron elevation in the metabolically critical intestinal cells being the primary contributor to the cause of death of aged C. elegans.

The formation of non-ferritin iron collections in HMW soluble- and insoluble forms precedes death, and is reminiscent of iron-filled intracellular inclusions, lipofuscin, neuromelanin and hemosiderin, which feature in the pathologies of aging and age-related disease, but are of questionable toxicity. We determined that the age-dependent accumulation of LMW-iron can be attenuated by SIH (19) to significantly delay aging in C. elegans. This result would be consistent with the accumulation of LMW-iron being a chemical mediator of senescence. Assessment of this species might reveal how preventing the age-dependent elevation of brain iron in calorically restricted primates preserves neuronal integrity (25). This iron species may also be a therapeutic target for disorders of advanced age e.g. nigral neuron loss in Parkinson's disease (26, 27).

### REFERENCES

1. Tatar M, Bartke A, & Antebi A (2003) The endocrine regulation of aging by insulin-like signals. Science 299(5611):1346-1351.
2. Kimura K D, Tissenbaum H A, Liu Y, & Ruvkun G (1997) daf-2, an insulin receptor-like gene that regulates longevity and diapause in Caenorhabditis elegans. Science 277(5328):942-946.
3. Kenyon C, Chang J, Gensch E, Rudner A, & Tabtiang R (1993) A C. elegans mutant that lives twice as long as wild type. Nature 366(6454):461-464.
4. Ogg S, et al. (1997) The Fork head transcription factor DAF-16 transduces insulin-like metabolic and longevity signals in C. elegans. Nature 389(6654):994-999.
5. James S A, et al. (2013) Direct in vivo imaging of essential bioinorganics in Caenorhabditis elegans. Metallomics: integrated biometal science 5(6):627-635.
6. McColl G, et al. (2012) Caenorhabditis elegans maintains highly compartmentalized cellular distribution of metals and steep concentration gradients of manganese. Plos One 7(2):e32685.
7. James S A, et al. (2013) Direct in vivo imaging of essential bioinorganics in Caenorhabditis elegans. Metallomics: integrated biometal science.
8. Meguro R, Asano Y, Iwatsuki H, & Shoumura K (2003) Perfusion-Perls and -Turnbull methods supplemented by DAB intensification for nonheme iron histochemistry: demonstration of the superior sensitivity of the methods in the liver, spleen, and stomach of the rat. Histochemistry and cell biology 120(1):73-82.
9. McGee M D, et al. (2011) Loss of intestinal nuclei and intestinal integrity in aging C. elegans. Aging cell 10(4):699-710.
10. Halliwell B & Gutteridge J M (1984) Oxygen toxicity, oxygen radicals, transition metals and disease. Biochem J 219(1):1-14.
11. McColl G, et al. (2008) Pharmacogenetic analysis of lithium-induced delayed aging in Caenorhabditis elegans. J Biol Chem 283(1):350-357.
12. Arosio P, Ingrassia R, & Cavadini P (2009) Ferritins: a family of molecules for iron storage, antioxidation and more. Biochim Biophys Acta 1790(7):589-599.
13. Gourley B L, Parker S B, Jones B J, Zumbrennen K B, & Leibold E A (2003) Cytosolic aconitase and ferritin are regulated by iron in Caenorhabditis elegans. J Biol Chem 278(5):3227-3234.
14. Kim Y I, Cho J H, Yoo O J, & Ahnn J (2004) Transcriptional regulation and life-span modulation of cytosolic aconitase and ferritin genes in C.elegans. J Mol Biol 342(2):421-433.
15. Cha'on U, et al. (2007) Disruption of iron homeostasis increases phosphine toxicity in Caenorhabditis elegans. Toxicological sciences: an official journal of the Society of Toxicology 96(1):194-201.
16. Budovskaya Y V, et al. (2008) An elt-3/elt-5/elt-6 GATA transcription circuit guides aging in C. elegans. Cell 134(2):291-303.
17. Ackerman D & Gems D (2012) Insulin/IGF-1 and hypoxia signaling act in concert to regulate iron homeostasis in Caenorhabditis elegans. PLoS genetics 8(3):e1002498.
18. McElwee J J, Schuster E, Blanc E, Thomas J H, & Gems D (2004) Shared transcriptional signature in Caenorhabditis elegans Dauer larvae and long-lived daf-2 mutants implicates detoxification system in longevity assurance. J Biol Chem 279(43):44533-44543.
19. Kalinowski D S & Richardson D R (2005) The evolution of iron chelators for the treatment of iron overload disease and cancer. Pharmacological reviews 57(4):547-583.
20. Reis-Rodrigues P, et al. (2012) Proteomic analysis of age-dependent changes in protein solubility identifies genes that modulate lifespan. Aging cell 11(1):120-127.
21. Smith A G, Carthew P, Francis J E, Edwards R E, & Dinsdale D (1990) Characterization and accumulation of ferritin in hepatocyte nuclei of mice with iron overload. Hepatology 12(6):1399-1405.
22. Kondo A, Deguchi J, & Okada S (1998) Intranuclear iron deposition in hepatocytes and renal tubular cells in mice treated with ferric nitrilotriacetate. Virchows Archiv: an international journal of pathology, 433(6):543-548.
23. Valentini S, et al. (2012) Manipulation of in vivo iron levels can alter resistance to oxidative stress without affecting ageing in the nematode C. elegans. Mechanisms of ageing and development 133(5):282-290.
24. Libina N, Berman J R, & Kenyon C (2003) Tissue-specific activities of C. elegans DAF-16 in the regulation of lifespan. Cell 115(4):489-502.
25. Kastman E K, et al. (2012) A calorie-restricted diet decreases brain iron accumulation and preserves motor performance in old rhesus monkeys. J Neurosci 32(34):11897-11904.
26. Lei P, et al. (2012) Tau deficiency induces parkinsonism with dementia by impairing APP-mediated iron export. Nat Med 18(2):291-295.
27. Kaur D, et al. (2003) Genetic or pharmacological iron chelation prevents MPTP-induced neurotoxicity in vivo: a novel therapy for Parkinson's disease. Neuron 37(6):899-909.
28. Wood W (1988) The Nematode Caenorhabditis elegans (Cold Spring Harbor Laboratory Press, Cold Spring Harbor, N.Y., USA).
29. Paterson D, et al. (2011) The X-ray Fluorescence Microscopy Beamline at the Australian Synchrotron. 10th International Conference on X-Ray Microscopy 1365:219-222.
30. Ryan C G, et al. (2010) The New Maia Detector System: Methods For High Definition Trace Element Imaging Of Natural Material. X-Ray Optics and Microanalysis, Proceedings 1221:9-17.
31. Ryan C G (2000) Quantitative trace element imaging using PIXE and the nuclear microprobe. Int J Imag Syst Tech 11(4):219-230.
32. Duce J A, et al. (2010) Iron-export ferroxidase activity of beta-amyloid precursor protein is inhibited by zinc in Alzheimer's disease. Cell 142(6):857-867.
33. Ye K, et al. (2010) Resveratrol attenuates radiation damage in Caenorhabditis elegans by preventing oxidative stress. Journal of radiation research 51(4):473-479.
34. Gomes A, Fernandes E, & Lima JLFC (2005) Fluorescence probes used for detection of reactive oxygen species. J Biochem Bioph Meth 65(2-3):45-80.
35. Bianchi L & Driscoll M (Culture of embryonic C. elegans cells for electrophysiological and pharmacological analyses. in WormBook, ed Community TCeR (Worm Book).
36. Dai Y, Whittal R M, & Li L (1999) Two-layer sample preparation: a method for MALDI-M S analysis of complex peptide and protein mixtures. Anal Chem 71(5):1087-1091.
37. Hare D J, et al. (2013) Profiling the iron, copper and zinc content in primary neuron and astrocyte cultures by rapid online quantitative size exclusion chromatography-inductively coupled plasma-mass spectrometry. Metallomics: integrated biometal science.
38. McColl G, et al. (2010) Insulin-like signaling determines survival during stress via posttranscriptional mechanisms in C. elegans. Cell metabolism 12(3):260-272.

The entire disclosures of all documents cited throughout this application are incorporated herein by reference.

1. A method of reducing senescence in a mammal by reducing the concentration of non-ferritin iron within the mammal, comprising the administration of a therapeutically effective amount of an iron chelator or an antioxidant, or a pharmaceutically acceptable salt thereof.

2. The method of claim 1, wherein the iron chelator is salicylaldehyde isonicotinoyl hydrazone (IH).

3. A method for extending the lifespan of a mammal, comprising the administration of a therapeutically effective amount of a scavenger of intracellular iron to facilitate extracellular clearance.

4. The method of claim 3, wherein the scavenger is an iron chelator or a pharmacetically acceptable salt thereof.

5. The method of claim 3, wherein the iron chelator is an acylhydrazone.

6. The method of claim 3, where the acylhydrazone is salicylaldehyde isonicotinoyl hydrazone (SIH).

7. The method of claim 3, wherein the lifespan of the mammal is extended by at least 10%, 20%, 30%, 40% or 50%.

8. A method for delaying the aging process caused by the accumulation of LMW-iron in a mammal comprising the administration of a therapeutically effective amount of a scavenger of intracellular iron to facilitate extracellular clearance.

9. The method of claim 8, wherein the scavenger is an iron chelator or a pharmaceutically acceptable salt thereof.

10. The method of claim 8, wherein the iron chelator is an acylhydrazone.

11. The method of claim 10, where the acylhydrazone is salicylaldehyde isonicotinoyl hydrazone (SIH).

12. A method of reducing the age-dependent accumulation of LMW-iron in a mammal, comprising the administration of a therapeutically effective amount of an acylhydrazone or a pharmaceutically acceptable salt thereof.

13. The method of claim 12, where the acylhydrazone is salicylaldehyde isonicotinoyl hydrazone (SIH).

14. The method of claim 12, wherein the accumulation of LMW-iron is the intracellular accumulation in the intestinal cells.

15. The method of claim 14, wherein the intracellular accumulation in the intestinal cells progresses from discrete vesicular to dispersed distribution.

16. The method of claim 12, where the accumulation of LMW-iron is the intracellular accumulation in the head region.

17. A method for reducing or eliminating the loss of iron homeostasis associated with the cause of aging in a mammal, the method comprising treating the mammal with a therapeutically effective amount of a scavenger of intracellular iron to facilitate extracellular clearance or prevent age-related toxic accumulation of iron.

18. The method of claim 17, wherein the scavenger is an iron chelator or a pharmaceutically acceptable salt thereof.

19. The method of claim 18, wherein the iron chelator is an acylhydrazone.

20. The method of claim 19, where the acylhydrazone is salicylaldehyde isonicotinoyl hydrazone.

21. A method for reducing the loss of ferritin sequestration of iron in a mammal, the method comprising treating the mammal with a therapeutically effective amount of a scavenger of intracellular iron to facilitate extracellular clearance or prevent age-related toxic accumulation of iron.

22. The method of claim 21, wherein the scavenger is an iron chelator or a pharmaceutically acceptable salt thereof.

23. The method of claim 22, wherein the iron chelator is an acylhydrazone.

24. The method of claim 23, where the acylhydrazone is salicylaldehyde isonicotinoyl hydrazone.

25. The method of claim 1, further comprising contacting the mammal with a FOXO transcription factor.

26. The method of claim 1, wherein the method results in an increase in the lifespan of the mammal by at least 10%, 20%, 30%, 40% or 50%.

27. A method for the treatment of an age-related disease in a mammal comprising the administration of a therapeutically effective amount of a compound that decreases the amount of LMW-iron in the mammal.

28. The method of claim 27, wherein the age-related disease is selected from the group consisting of heart diseases, cancer, Alzheimer's disease, and arthritis.

29. A method for the treatment of senescence or a disease of old age in a mammal comprising administrating a therapeutically effective amount of a compound that lowers the LMW-iron concentration in the mammal.
